(19) United States
(12) Patent Application Publication
(10) Pub. No.: US 2012/0225180 A1
(43) Pub. Date: Sep. 6, 2012

(54) SALTINESS-IMPROVING AGENT FOR SALTY FOOD AND DRINK

(76) Inventors: Yoshiko Kurobayashi, Kanagawa (JP); Setsuko Nakai, Kanagawa (JP); Kikue Kubota, Tokyo (JP)

(21) Appl. No.: 13/509,392

(22) PCT Filed: Nov. 12, 2010
(86) PCT No.: PCT/JP2010/070169

§ 371 Date: May 11, 2012

(30) Foreign Application Priority Data
Nov. 12, 2009 (JP) 2009-259307
May 18, 2010 (JP) 2010-114364

(51) Int. Cl. A23L 1/226; C07D 307/87

(52) U.S. Cl. 426/536; 549/307

(57) ABSTRACT

[Object] To improve saltiness, for example, to reduce feeling of stimulation of saltiness of salty food and drink, without imparting unnecessary taste or aroma to the food and drink.

[Solution] A saltiness-improving agent for salty food and drink includes phthalides as an active ingredient. Addition of a very small amount of phthalides that are not sensed as aroma to food and drink improves undesirable saltiness of various salty food and drink products and enhances or provides pleasant saltiness.

### TECHNICAL FIELD

The present invention relates to a saltiness-improving agent for salty food and drink and, more specifically, relates to a saltiness-improving agent containing phthalides as an active ingredient for improving the saltiness of salty food and drink to provide preferred saltiness.

### BACKGROUND ART

Saltiness is usually caused by sodium chloride and is not contained in a large amount in natural raw materials, that is, in uncooked food materials themselves. Accordingly, we modern people impart palatability to food by adding salt purified from, for example, seawater or rock salt to food materials and cooking them, but in some cases, the saltiness is felt to be acute or stimulative by compatibility with the food raw materials. Accordingly, in order to reduce the stimulation of saltiness, various additives have been proposed. For example, a method of reducing the stimulation of saltiness by adding dihydrochalcones to salty food and drink (Patent Literature 1), a method of adding ethyl-α-glucoside (Patent Literature 2), a method of improving saltiness and bitterness of, for example, food by adding thaumatin and phenoxyalkanoic acid (Patent Literature 3), and improvement of saltiness by adding pyroglutamate (Patent Literature 4) have been proposed.

### CITATION LIST

### Patent Literature

Patent Literature 1: Japanese Patent Laid-Open No. Sho 50-13568

Patent Literature 2: Japanese Patent Laid-Open No. Hei 4-112766

Patent Literature 3: Japanese Patent Laid-Open No. Hei 7-79730

Patent Literature 4: Japanese Patent Laid-Open No. 2001-299266

### SUMMARY OF INVENTION

### Technical Problem

In the above-mentioned proposals relating to the reduction in feeling of stimulation of saltiness, unnecessary sweetness or taste is added, or the effect is insufficient. Accordingly, it is demanded to effectively improve the saltiness to naturally harmonious saltiness without causing any off-taste or off-flavor by using an ingredient originated from food and drink.

### Solution of Problem

The present inventors have diligently studied for improvement of saltiness of food and drink and, as a result, have found that an ingredient derived from Umbelliferae plants is involved in enhancement of, for example, savor and flavor of chicken broth and that the ingredient is phthalides (J. Agric. Food Chem., Vol. 56, No. 2, 512-516, 2008). Accordingly, the present inventors have assumed that phthalides further have various taste-improving effects and have carried out further studies. As a result, surprisingly, it has been found that mere addition of a very small amount of phthalides that are not sensed as aroma to food and drink improves undesirable saltiness of various food and drink products and enhances or provides pleasant saltiness. Thus, the present invention has been accomplished.

Accordingly, the present invention provides the following aspects:

(1) a saltiness-improving agent for salty food and drink, the agent including phthalides as an active ingredient;
(2) the saltiness-improving agent according to aspect (1), wherein the improvement of saltiness is a reduction in feeling of stimulation of saltiness;
(3) the saltiness-improving agent according to aspect (1) or (2), wherein the phthalides are at least one selected from sedanenolide, sedanolide, 3-n-butylphthalide, and 3-butylidenephthalide;
(4) a saltiness-improving agent composition including the saltiness-improving agent for salty food and drink according to any one of aspects (1) to (3) in an amount of 10 ppb to 1% as phthalides;
(5) a method of improving saltiness of salty food and drink, the method including adding the saltiness-improving agent according to any one of aspects (1) to (3) to the salty food and drink in an amount of 0.01 ppb to 10 ppm as phthalides; and
(6) a method of improving saltiness of salty food and drink, the method including adding the saltiness-improving agent composition according to aspect (4) to the salty food and drink in an amount of 0.01 ppb to 10 ppm as phthalides.

### Advantageous Effects of Invention

According to the present invention, it is possible to improve saltiness of salty food and drink by, for example, reducing the feeling of stimulation of saltiness without imparting unnecessary taste or aroma to the food and drink.

### DESCRIPTION OF EMBODIMENTS

The phthalides used in the present invention refer to a compound with a phthalide skeleton among a group of compounds characteristically existing in essential oil of Umbelliferae plants and having characteristic spicy herbal medicine smell. Specifically, the phthalides refer to, for example, sedanenolide, sedanolide, 3-n-butylphthalide, 3-butylidenephthalide, ligustilide, cnidilide, isocnidilide, neocnidilide, methyl sedanoate, and 3-butylhexahydrophthalide. Among these compounds, particularly preferred are sedanenolide, sedanolide, 3-n-butylphthalide, and 3-butylidenephthalide. The phthalides may be synthetic products or may be prepared by obtaining an extract or essential oil from an Umbelliferae plant by, for example, water or solvent extraction or steam distillation and purifying the extract or essential oil by, for example, various known methods.

These compounds may be used alone or in a combination of two or more thereof. Furthermore, essential oil of for example. Cnidium rhizome, Angelica sinensis, Lavage, or celery, or an extract, such as oleoresin, containing the above-mentioned compounds may be used directly, or the compounds may be used as an ingredient contained in a flavor preparation.

Incidentally, essential oil and oleoresin of Umbelliferae plants are natural raw materials and are preferred as the saltiness-improving agent of the present invention from the viewpoint of safety and relief. However, the essential oil or oleoresin contains, in addition to the above-mentioned phthalides, hydrocarbons such as limonene, myrcene, β-caryophyllene, α-selinene, β-selinene, and γ-selinene and has characteristic greenish aroma. Such greenish aroma is pleasant savor when the essential oil or oleoresin is used for providing the savor of Umbelliferae plants, as in usual flavoring. However, in the use as the saltiness-improving agent of the present invention, these hydrocarbons unnecessarily provide the greenish aroma and are therefore preferable to be reduced or removed. In particular, selinenes have strong greenish aroma specific to Umbelliferae plants and are thus desired to be reduced or removed as much as possible. Among these hydrocarbons, substances having a relatively low boiling point such as limonene (boiling point: 176° C., atmospheric pressure) are easily reduced by distillation (temperature: 50 to 70° C., pressure: 500 to 1000 Pa) using, for example, a usual rectifying column. Selinenes (β-selinene, boiling point: 260° C., atmospheric pressure), which are sesquiterpene hydrocarbons, and phthalides (sedanenolide, boiling point: 367° C., atmospheric pressure) both belong to a group having relatively high boiling points as those of flavoring compounds, and these compounds therefore remain in the still residue in distillation using a usual rectifying column, and it is difficult to separate these compounds. Therefore, it is necessary to remove selinenes from essential oil or oleoresin of Umbelliferae plants by a simple and low-cost method.

The hydrocarbons including selinenes can be reduced or removed from essential oil or oleoresin of Umbelliferae plants by various types of chromatography, but as a simple and industrially practical method, molecular distillation can be recommended. The molecular distillation is performed under high vacuum conditions and is a purification method by distillation in such a manner that evaporated molecules arrive and are condensed at a cooling surface without causing collision with other molecules. Any type of apparatus or instrument that can be applied to this method can be used without particular limitation, and a batch or continuous molecular distillation apparatus is usually used and is classified based on the system into a falling film molecular distillation apparatus and a centrifugal film molecular distillation apparatus. Among these apparatuses, from the viewpoint of forming stable thin-film conditions, the centrifugal film molecular distillation apparatus, in particular, a continuous-type centrifugal film molecular distillation apparatus is preferably used.

The conditions for removing the hydrocarbons with a centrifugal film molecular distillation apparatus are, for example, a temperature of 90 to 110° C. and a pressure of 10 to 30 Pa. Under such conditions, not only hydrocarbons having low boiling points such as limonene but also selinenes, which are sesquiterpene hydrocarbons, can be distilled away, and phthalides can be obtained as a still residue. In the case of subjecting essential oil as a raw material to molecular distillation, one-time molecular distillation can provide the saltiness-improving agent of the present invention as the still residue containing 60% or more of phthalides. Furthermore, in the case of a raw material containing nonvolatile components such as oleoresin, after removal of hydrocarbons under the above-mentioned conditions, the still residue containing phthalides is subjected to molecular distillation again to evaporate phthalides under conditions of a temperature of 140 to 160° C. and a pressure of 10 to 30 Pa to provide the saltiness-improving agent of the present invention as the distillate containing 60% or more of phthalides.

Incidentally, the molecular distillation itself is generally used not only for purification of, for example, oils and fats but also for removing the terpene fraction from essential oil of, for example, orange. However, it is not known to apply the molecular distillation for reducing or removing unnecessary components such as limonene and selinenes from essential oil or oleoresin of Umbelliferae plants to purify a saltiness-improving agent.

The present invention relates to a saltiness-improving agent for salty food and drink, including phthalides as the active ingredient, and relates to a saltiness-improving agent that can improve saltiness of salty food and drink by adding these phthalides to the salty food and drink in such a low amount that is not sensed as spicy herbal medicine smell specific to the phthalides.

Next, the present invention will be described with reference to individual embodiments below.

In one embodiment, the present invention provides a saltiness-improving agent for salty food and drink. The salty food and drink refers to food and drink having saltiness, contains salt, that is, sodium chloride, and means food and drink that tastes salty. However, if the salt concentration is too low, saltiness itself is hardly felt. Accordingly, the salty food and drink generally refers to food and drink containing 0.1% by mass or more of salt and giving the feeling of stimulation of saltiness. Conventionally, salt is used not only for obtaining saltiness but also for food storage, and such food must maintain its high salt concentration for, for example, suppressing proliferation of microorganisms. In addition, even in food and drink of which salt concentration is not very high, saltiness accompanied by stimulation is felt in some cases.

Examples of the sally food and drink include various kinds of Japanese confectionery such as okaki (a rice cracker) and senbei (a flat, rounded rice cracker); various kinds of Western confectionery such as salt caramel, salt candy, cookies, and bread; various kinds of snack food such as potato chips; various kinds of paste such as flower paste and peanut paste; Japanese pickles, tsukudani (food boiled down in soy sauce), and shiokara (salted fish); various kinds of preserved meat products such as ham, sausage, bacon, dried sausage, and beef jerky; various kinds of seafood products such as fish ham, fish sausage, kamaboko (boiled fish paste), chikuwa (tubular fish paste), hanpen (steamed fish paste), and tempura (deep-fried fish and vegetables); various curries such as instant curry, retort-pouch curry, and canned curry; various kinds of seasoning such as miso (soybean paste), powdered miso, soy sauce, powdered soy sauce, unrefined soy sauce, fish sauce, sauce, broth, ketchup, mayonnaise, dressing, solid bouillon, grilled meat sauce, curry roux, stew roux, soup stock, and dashi (Japanese soup stock); and miso soup, soba soup, Japanese dear soup, consomme soup, ramen soup, and dressing.

In the salty food and drink of the present invention, the blending ratio of the phthalides in the final product is preferably 0.01 ppb to 10 ppm, more preferably 0.1 ppb to 1 ppm. Furthermore, in many cases, practically, these phthalides are added to a formulation, and the formulation is added to a final product. In such a case, since the amount of the formulation added to the final product is about 0.01 to 1%, the amount of the phthalides added to the formulation is 10 ppb to 1%, more preferably 100 ppb to 1000 ppm. In this blending ratio, the feeling of stimulation of saltiness of salty food and drink is reduced, and the saltiness accompanied by the feeling of stimulation caused by salt is improved to mild saltiness.

The concentration of phthalides to be added for improving saltiness of food and drink is as described above, and even if the concentration of the phthalides is higher than the above-mentioned range, the effect of improving saltiness is not increased. Accordingly, it is not necessary to add the phthalides in an amount exceeding the above-mentioned range. However, if desired, for example, in the case of imparting the aroma specific to phthalides to food and drink, the phthalides may be added to the food and drink in an amount exceeding the above-mentioned range.

The present invention will be more specifically described by the following examples, but is not limited thereto.

### EXAMPLES

### Example 1

### Reduction of Stimulation of Saltiness of Shiokara (Salted Fish)

Sedanenolide was added as a saltiness-improving agent (salty stimulation-reducing agent) of the present invention to commercially available salted squid to obtain shiokara containing sedanenolide at the concentrations shown in the following Table 1.

### Comparison of Flavor

Table 1 shows the comparison evaluation of flavor by 10 panelists for the shiokara containing or not containing the product of the present invention.

TABLE 1
Saltiness evaluation of shiokara

| Concentration of | | Evaluation | | | Flavor | |
|---|---|---|---|---|---|---|
| sedanenolide | | A | B | N | judgment | Note |
| Sedanenolide-free | | | | | | |
| 1 | ppt | 10 | 0 | 0 | 0 | |
| 0.01 | ppb | 6 | 4 | 0 | 0 | |
| 0.1 | ppb | 2 | 7 | 1 | 0 | |
| 1 | ppb | 3 | 2 | 5 | 0 | |
| 0.01 | ppm | 2 | 2 | 6 | 3 | |
| 0.1 | ppm | 1 | 2 | 7 | 4 | |
| 1 | ppm | 0 | 1 | 9 | 2 | |
| 10 | ppm | 0 | 1 | 9 | 1 | slight spicy aroma |
| | | | | | | was sensed |

### Evaluation

A: the number of panelists who felt stimulation of saltiness
B: the number of panelists who slightly felt stimulation of saltiness
N: the number of panelists who judged that the stimulation of saltiness was reduced to mild saltiness
Flavor judgment: the number of panelists who judged it as most proper saltiness

As shown in Table 1, it was judged that though shiokara not containing sedanenolide had feeling of stimulation of saltiness, the stimulation of saltiness was improved in the shiokara containing 0.01 ppb to 10 ppm of sedanenolide. When the amount of sedanenolide was about 0.1 ppb to 1 ppm, particularly good results were obtained. In an amount of 10 ppm, though the stimulation of saltiness itself was improved to mild saltiness, a slight spicy aroma other than saltiness was also felt. Even in a low concentration of 0.01 ppb, some panelists felt some improvement in stimulation of saltiness. Thus, it was revealed that the effect of improving saltiness can be obtained even at a low concentration.

### Reference Example 1

### Purification of Essential Oil by Precision Distillation

As a commercially available celery seed essential oil (Reference Product 1), an essential oil having a component composition composed of 1.86% of 3-n-butylphthalide, 5.4% of sedanenolide, 0.32% of sedanolide, 66.2% of limonene, 12.9% of β-selinene, and 13.3% of others (measured by gas chromatography) was used. Reference Product 1 (120.5 g) was subjected to reduced pressure precision distillation under the following conditions. The pressure was gradually reduced from 1000 Pa to 500 Pa at an internal temperature of 54° C., and when distillation of distillate stopped, the precision distillation was terminated to obtain a distillate and a still residue (Reference Product 2). The compositions of the resulting distillate and still residue were as follows: 80.3 g of the distillate portion was composed of 1.43% of 3-n-butylphthalide, 3.72% of sedanenolide, 0.27% of sedanolide, 85.5% of limonene, 3.5% of β-selinene, and 5.58% of others, and 40.8 g of the still residue portion (Reference Product 2) was composed of 7.07% of 3-n-butylphthalide, 20.5% of sedanenolide, 1.22% of sedanolide, 0% of limonene, 43.4% of β-selinene, and 27.81% of others).

As described above, in the precision distillation, all of limonene was evaporated, and 85% or more of the distillate portion was limonene. On the other hand, though the still residue portion contained concentrated phthalides, it was recognized that β-selinene was also concentrated in the still residue portion.

### Example 2

### Purification of Oleoresin by Molecular Distillation

As a commercially available celery oleoresin (Reference Product 3), oleoresin having volatile component composition composed of 5.0% of 3-n-butylphthalide, 41.3% of sedanenolide, 2.5% of sedanolide, 30.4% of limonene, 8.2% of β-selinene, and 12.6% of others was used.

To Reference Product 3 (362.6 g), 72.9 g (20% of Reference Product 3) of rice salad oil was added. The resulting mixture was sufficiently mixed and fed to a centrifugal film molecular distillation apparatus. Thin film distillation was performed by allowing the mixture to flow into a heat transfer surface from a feed nozzle at a rate of 3.6 g/min under a reduced pressure of 15 to 28 Pa, allowing cooling water to flow in a condenser, and cooling a cold trap with dry ice/acetone. On this occasion, the treatment liquid was heated under conditions of a temperature of 100° C. for about 1.0 sec for a thin film having a thickness of 0.1 mm on average. After distillation, 25.1 g of a cold trap portion, 10.6 g of a cooling water trap portion, and 399.8 g of a still residue portion were obtained.

The volatile component composition of the still residue portion was 6.9% of 3-n-butylphthalide, 58.3% of sedanenolide, 3.0% of sedanolide, 8.3% of limonene, 13.2% of β-selinene, and 10.3% of others).

The still residue portion (399.8 g) was fed to the centrifugal film molecular distillation apparatus again, and thin film distillation was performed by allowing the still residue to flow into the heat transfer surface from the feed nozzle at a rate of 3.6 g/min under a reduced pressure of 15 to 28 Pa, allowing cooling water to flow in the condenser, and cooling the cold trap with dry ice/acetone. On this occasion, the treatment liquid was heated under conditions of a temperature of 150° C. for about 1.0 sec for a thin film having a thickness of 0.1 mm on average. Only the distillation portion after distillation was used as Invention Product 1 (yield: 54.7 g).

The component composition of Invention Product 1 was 8.0% of 3-n-butylphthalide, 65.5% of sedanenolide, 3.5% of sedanolide, 0.6% of limonene, 10.9% of β-selinene, and 11.5% of others. The still residue portion (345.1 g) of the second molecular distillation hardly contained volatile components that can be detected by gas chromatography.

### Example 3

### Preparation of Saltiness-improving Agent Powder

An oil phase portion was prepared by mixing and dissolving 19.6 g of medium chain fatty acid triglyceride and 30 g of SAIB in 0.093 g of 3-n-butylphthalide, 0.27 g of sedanenolide, and 0.016 g of sedanolide. Separately, 955 g of Pinedex No. 2 and 50 g of sucrose fatty acid ester having an HLB of 15 were added and dissolved in 680 g of soft water, and the resulting solution was heat-sterilized at 85° C. for 15 min. This solution was cooled to about 40° C., and 50 g of the oil phase portion prepared above was then poured thereto while stirring with a TK-homomixer (manufactured by Tokusyu Kika Kogyo Co., Ltd.). The resulting mixture was further stirred at 5000 rpm for 5 min for emulsification to obtain 1690 g of emulsion. This emulsion was dried using a spray dryer (manufactured by Niro A/S, Mobile Minor) at a blast temperature of 150° C. and at an exhaust temperature of 80° C. to obtain 900 g of saltiness-improving agent powder (Invention Product 2: containing 0.0093% of 3-n-butylphthalide, 0.027% of sedanenolide, and 0.0016% of sedanolide).

1. A saltiness-improving agent for salty food, the agent comprising phthalides as an active ingredient, wherein
the salty food is food selected from the group consisting of Japanese confectionery, Western confectionery, snack food, flower paste, peanut paste, Japanese pickles, tsukudani (food boiled down in soy sauce), shiokara (salted fish), ham, sausage, bacon, dried sausage, beef jerky, fish ham, fish sausage, kamaboko (boiled fish paste), chikuwa (tubular fish paste), hanpen (steamed fish paste), curries, miso (soybean paste), powdered miso, soy sauce, powdered soy sauce, unrefined soy sauce, fish sauce, sauce, ketchup, mayonnaise, dressing, and grilled meat sauce.

2. The saltiness-improving agent according to claim 1, wherein the improvement of saltiness is a reduction in feeling of stimulation of saltiness.

3. The saltiness-improving agent according to claim 1, wherein the phthalides are at least one selected from sedanolide, sedanolide, 3-n-butylphthalide, and 3-butylidenephthalide.

4. A saltiness-improving agent composition for salty food, the composition comprising the saltiness-improving agent for salty food according to claim 1 in an amount of 10 ppb to 1% as phthalides, wherein
the salty food is food selected from the group consisting of Japanese confectionery, Western confectionery, snack food, flower paste, peanut paste, Japanese pickles, tsukudani (food boiled down in soy sauce), shiokara (salted fish), ham, sausage, bacon, dried sausage, beef jerky, fish ham, fish sausage, kamaboko (boiled fish paste), chikuwa (tubular fish paste), hanpen (steamed fish paste), curries, miso (soybean paste), powdered miso, soy sauce, powdered soy sauce, unrefined soy sauce, fish sauce, sauce, ketchup, mayonnaise, dressing, and grilled meat sauce.

5. A method of improving saltiness of salty food, the method comprising adding the saltiness-improving agent according to claim 1 to the salty food in an amount of 0.01 ppb to 10 ppm as phthalides, wherein
the salty food is food selected from the group consisting of Japanese confectionery, Western confectionery, snack food, flower paste, peanut paste, Japanese pickles, tsukudani (food boiled down in soy sauce), shiokara (salted fish), ham, sausage, bacon, dried sausage, beef jerky, fish ham, fish sausage, kamaboko (boiled fish paste), chikuwa (tubular fish paste), hanpen (steamed fish paste), curries, miso (soybean paste), powdered miso, soy sauce, powdered soy sauce, unrefined soy sauce, fish sauce, sauce, ketchup, mayonnaise, dressing, and grilled meat sauce.

6. A method of improving saltiness of salty food, the method comprising adding the saltiness-improving agent composition according to claim 4 to the salty food in an amount of 0.01 ppb to 10 ppm as phthalides, wherein
the salty food is food selected from the group consisting of Japanese confectionery, Western confectionery, snack food, flower paste, peanut paste, Japanese pickles, tsukudani (food boiled down in soy sauce), shiokara (salted fish), ham, sausage, bacon, dried sausage, beef jerky, fish ham, fish sausage, kamaboko (boiled fish paste), chikuwa (tubular fish paste), hanpen (steamed fish paste), curries, miso (soybean paste), powdered miso, soy sauce, powdered soy sauce, unrefined soy sauce, fish sauce, sauce, ketchup, mayonnaise, dressing, and grilled meat sauce.
